(19) United States
(12) Patent Application Publication
(10) Pub. No.: US 2025/0375127 A1
(43) Pub. Date: Dec. 11, 2025

(54) GLYCATED HEMOGLOBIN PERCENTAGE DETECTION METHOD AND ELECTRONIC DEVICE USING THE SAME

(71) Applicant: NATIONAL TAIWAN UNIVERSITY, Taipei City (TW)

(72) Inventors: CHI-KUANG SUN, Taipei City (TW); PENG-LI WANG, Taipei City (TW); TZUNG-DAU WANG, Taipei City (TW)

(21) Appl. No.: 18/940,861

(22) Filed: Nov. 8, 2024

(30) Foreign Application Priority Data
Jun. 7, 2024 (TW) 113121138

(51) Int. Cl. A61B 5/1455; A61B 5/00

(52) U.S. Cl. CPC A61B 5/1455; A61B 5/6802

(57) ABSTRACT

A glycated hemoglobin percentage detection method includes: obtaining optical response information of a blood under test; performing a normalization and correction process based on the optical response information of the blood under test of a first wavelength to obtain normalized optical response information of the blood under test; selecting normalized optical response information of a second wavelength and normalized optical response information of a third wavelength from the normalized optical response information of the blood under test for analysis to determine a glycated hemoglobin percentage in the blood under test.

### CROSS-REFERENCE TO RELATED PATENT APPLICATION

This application claims the benefit of priority to Taiwan Patent Application No. 113121138, filed on Jun. 7, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a blood component percentage detection method and an electronic device, and more particularly to a low-cost glycated hemoglobin percentage detection method and an electronic device using the method.

### BACKGROUND OF THE DISCLOSURE

Most blood component detection methods or blood component detection devices require complicated detection processes and apparatuses, and mostly require invasive acquisition of blood under tests. When the blood component to be detected is glycated hemoglobin, the measurement of the glycated hemoglobin is used in the detection process that mainly includes immunoturbidimetric method, boric acid affinity chromatography, high performance liquid chromatography (HPLC), and enzymatic method. The disadvantages of these processes include invasiveness, long processing time, high cost, reliance on and limitations on professional apparatuses, and inconvenience for detection.

Furthermore, according to U.S. Patent Application Publication Ser. No. 2021/0307661, detection of spectral information is performed by using the concentration of two different blood components, oxygenated protein and deoxygenated protein, and the concentration of a predetermined component (e.g., glycated hemoglobin) cannot be individually detected.

### SUMMARY OF THE DISCLOSURE

In order to solve the above-mentioned problems, one of the technical aspects adopted by the present disclosure is to provide a glycated hemoglobin percentage detection method. The glycated hemoglobin percentage detection method includes: obtaining optical response information of a blood under test; performing a normalization and correction process based on the optical response information of the blood under test of a first wavelength to obtain normalized optical response information of the blood under test; selecting normalized optical response information of a second wavelength and normalized optical response information of a third wavelength from the normalized optical response information of the blood under test for analysis to determine a glycated hemoglobin percentage in the blood under test.

In order to solve the above-mentioned problems, another one of the technical aspects adopted by the present disclosure is to provide an electronic device using a glycated hemoglobin percentage detection method for detecting a glycated hemoglobin percentage of a blood under test. The electronic device includes a controller, a storage circuit connected to the controller, a first wavelength light source providing circuit, a second wavelength light source providing circuit, a third wavelength light source providing circuit, a first wavelength optical response information receiver, a second wavelength optical response information receiver, and a third wavelength optical response information receiver. The storage circuit is connected to the controller. The first wavelength light source providing circuit is connected to the controller. The second wavelength light source providing circuit is connected to the controller. The third wavelength light source providing circuit is connected to the controller. The first wavelength optical response information receiver is connected to the controller. The second wavelength optical response information receiver is connected to the controller. The third wavelength optical response information receiver is connected to the controller. When the first wavelength light source providing circuit emits light of a first wavelength to a blood under test, the second wavelength light source providing circuit emits light of a second wavelength to the blood under test, and the third wavelength light source providing circuit emits light of a third wavelength to the blood under test, the first wavelength optical response information receiver receives optical response information of the blood under test corresponding to the first wavelength, the second wavelength optical response information receiver receives optical response information of the blood under test corresponding to the second wavelength, and the third wavelength optical response information receiver receives optical response information of the blood under test corresponding to the third wavelength. The controller performs a normalization and correction process according to the optical response information corresponding to the first wavelength, and the controller performs the normalization and correction process for the optical response information corresponding to the second wavelength and the optical response information corresponding to the third wavelength according to the optical response information corresponding to the first wavelength, to obtain a normalized optical response information corresponding to the second wavelength and a normalized optical response information corresponding to the third wavelength for the blood under test. The controller analyzes the normalized optical response information corresponding to the second wavelength and the normalized optical response information corresponding to the third wavelength to determine a glycated hemoglobin percentage of the blood under test.

One of the beneficial effects of the present disclosure is that the blood component percentage detection method and electronic device provided by the present disclosure can use common ultraviolet and visible light wavelength ranges for spectral detection, and simplify the blood component percentage comparison process, not only This effectively reduces costs and can significantly reduce the amount of calculations required by the electronic device to determine the percentage of blood components.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of a glycated hemoglobin percentage detection method according to a first embodiment of the present disclosure;

FIG. 2A is a schematic diagram of spectral analysis using standard samples and clinical samples according to the first embodiment of the present disclosure;

FIG. 2B is another schematic diagram of spectral analysis using the standard samples and the clinical samples according to the first embodiment of the present disclosure;

FIG. 3A is a schematic diagram of a light source providing circuit and a spectrum detection circuit illuminate a blood under test to obtain optical response information according to the first embodiment of the present disclosure;

FIG. 3B is another schematic diagram of the light source providing circuit and the spectrum detection circuit illuminating the blood under test to obtain optical response information according to the first embodiment of the present disclosure;

FIG. 4 is a schematic diagram of optical response information of the standard samples and the clinical samples;

FIG. 5 is a schematic diagram of optical response information of glycated hemoglobin standard samples having a same concentration and different percentages;

FIG. 6A is a schematic diagram of determining a second wavelength and a third wavelength by using absorption characteristics of optical response information at different wavelengths;

FIG. 6B is another schematic diagram of determining the second wavelength and the third wavelength by using the absorption characteristics of the optical response information at different wavelengths;

FIG. 7 is a schematic diagram of a user wearing an electronic device according to a fifth embodiment of the present disclosure; and

FIG. 8 is a functional block diagram of the electronic device according to the fifth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

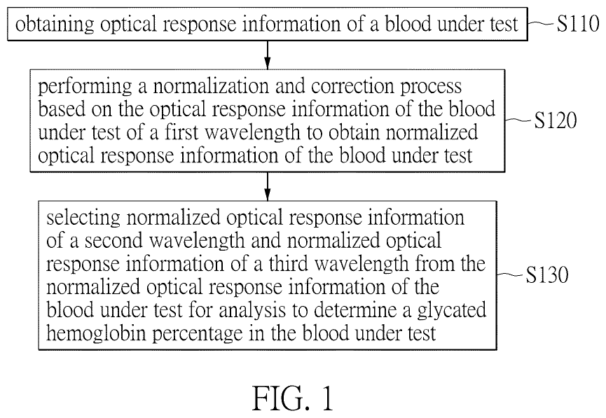

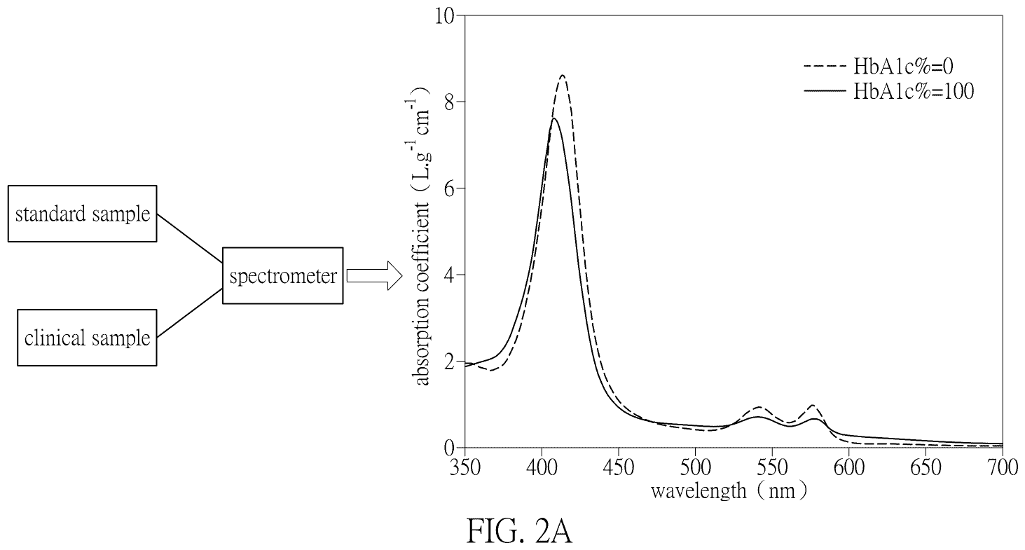

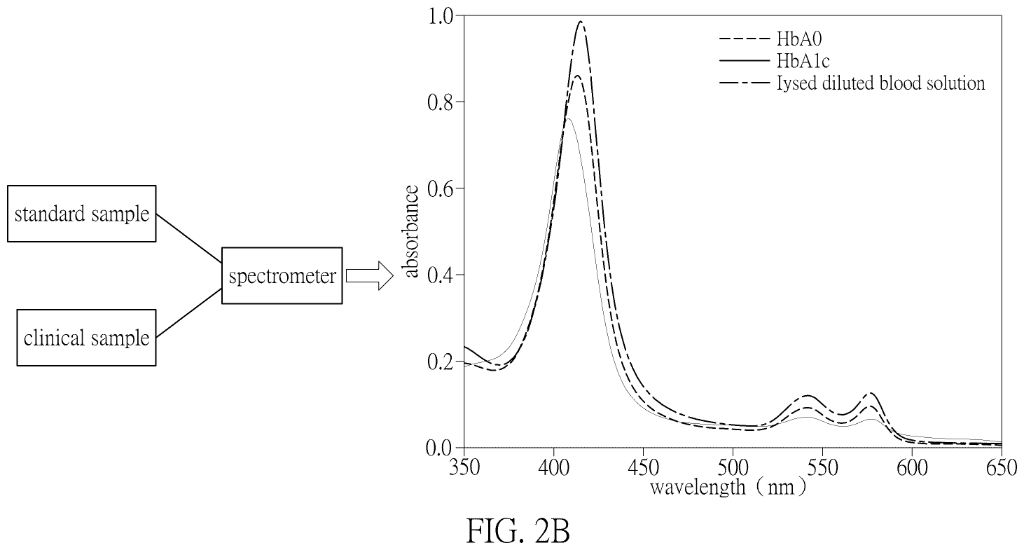

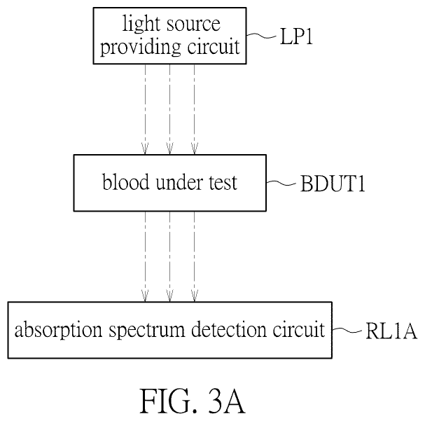

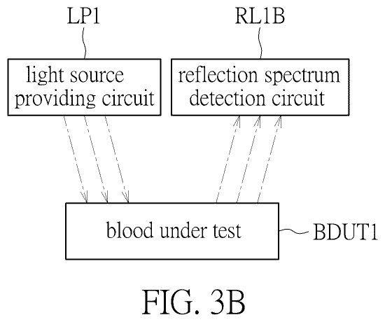

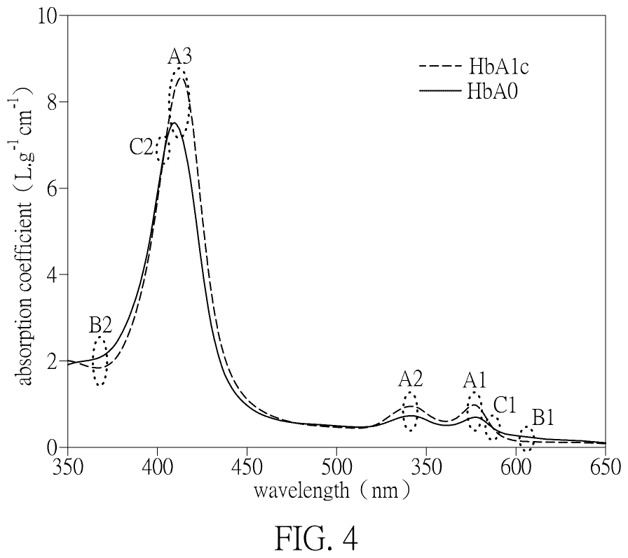

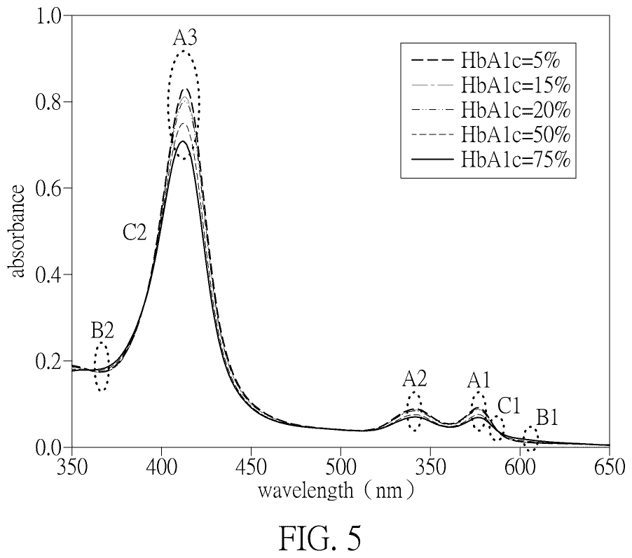

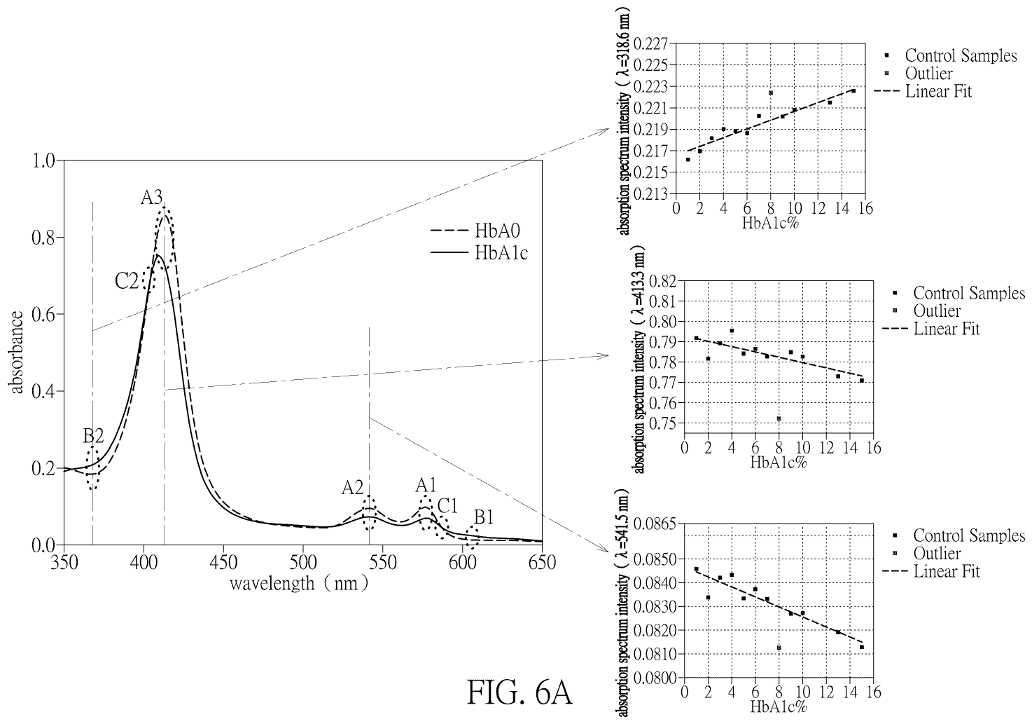

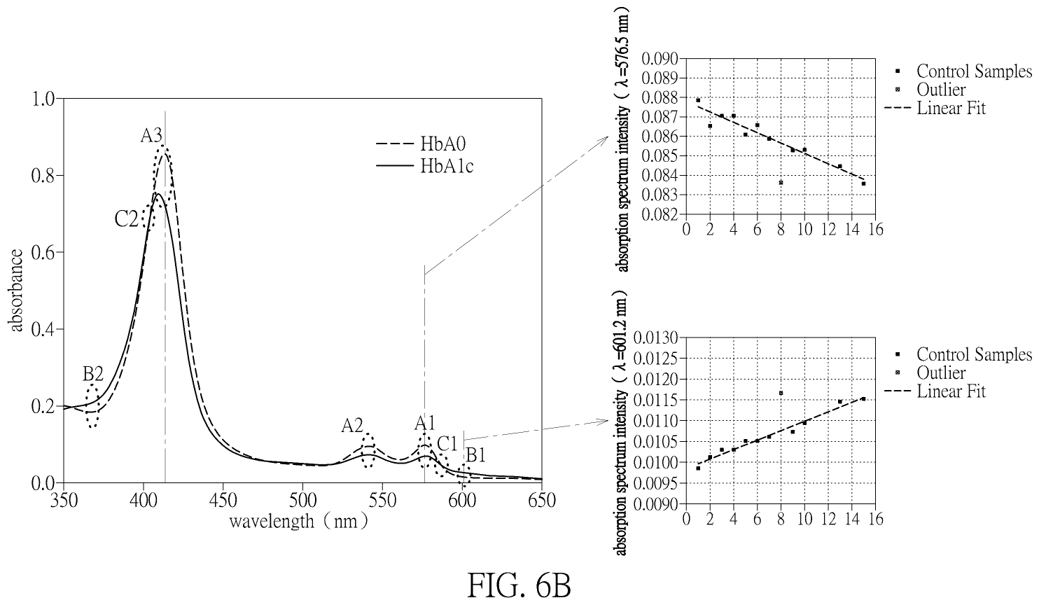

Referring to FIG. 1, FIG. 2A, FIG. 2B, FIG. 3A, FIG. 3B, FIG. 4, FIG. 5, FIG. 6A and FIG. 6B, FIG. 1 is a flowchart of a glycated hemoglobin percentage detection method according to a first embodiment of the present disclosure, FIG. 2A is a schematic diagram of spectral analysis using standard samples and clinical samples according to the first embodiment of the present disclosure, FIG. 2B is another schematic diagram of spectral analysis using the standard samples and the clinical samples according to the first embodiment of the present disclosure, FIG. 3A is a schematic diagram of a light source providing circuit and a spectrum detection circuit illuminating a blood under test to obtain optical response information according to the first embodiment of the present disclosure, FIG. 3B is another schematic diagram of the light source providing circuit and the spectrum detection circuit illuminating the blood under test to obtain optical response information according to the first embodiment of the present disclosure, FIG. 4 is a schematic diagram of optical response information of the standard samples and the clinical samples, FIG. 5 is a schematic diagram of optical response information of glycated hemoglobin standard samples having a same concentration and different percentages, FIG. 6A is a schematic diagram of determining a second wavelength and a third wavelength by using absorption characteristics of optical response information at different wavelengths, and FIG. 6B is another schematic diagram of determining the second wavelength and the third wavelength by using the absorption characteristics of the optical response information at different wavelengths.

In this embodiment, a glycated hemoglobin percentage detection method is provided. The glycated hemoglobin percentage detection method of this embodiment is suitable for an electronic device (not shown). The electronic device (not shown) will be described in more detail in the fifth embodiment. The electronic device (not shown) can be a smartphone, a tablet, a wearable electronic device, a medical device, or a non-invasive medical instrument. In addition, the electronic device (not shown) can also be a spectrometer, and a blood under test BDUT1 is a blood sample. The blood under test is a blood content extracted from a user.

The glycated hemoglobin percentage detection method in this embodiment includes following steps.
Step S110 includes: obtaining optical response information of a blood under test.
Step S120 includes: performing a normalization and correction process based on the optical response information of the blood under test of a first wavelength to obtain normalized optical response information of the blood under test.
Step S130 includes: selecting normalized optical response information of a second wavelength and normalized optical response information of a third wavelength from the normalized optical response information of the blood under test for analysis to determine a glycated hemoglobin percentage in the blood under test.

In step S110 and step S120, reference is simultaneously made to FIGS. 2A, 2B, 3A, and 3B. The glycated hemoglobin percentage detection method in this embodiment uses a light source providing circuit LP1 as shown in FIG. 3A or 3B and a spectrum detection circuit (that is implemented in two forms: an absorption spectrum detection circuit RL1A in FIG. 3A and a reflection spectrum detection circuit RL1B in FIG. 3B) that detects the blood under test BDUT1. In an existing detection process of the blood under test BDUT1 as shown in FIG. 2A, standard samples and clinical samples are sent to the spectrometer for detection to obtain optical response information for analysis. The glycated hemoglobin percentage detection method in this embodiment also uses a similar process, but the analysis process of the glycated hemoglobin percentage detection method in this embodiment is as follows. The blood under test BDUT1 may be a blood sample extracted from the user, as shown in FIG. 2A and FIG. 2B. The blood under test BDUT1 may also be a blood content stored in the body of the user. That is, the light source providing circuit LP1 and the spectrum detection circuit (the absorption spectrum detection circuit RL1A or the reflection spectrum detection circuit RL1B) of this embodiment are used to externally illuminate human body parts, such as but not limited to, fingers and wrists, so as to obtain reflected light that is then analyzed.

Firstly, reference is made to FIG. 3A, FIG. 3B, and FIG. 4. The light source providing circuit LP1 can provide light of a wavelength range or three wavelengths to illuminate the blood under test BDUT1, such that the blood under test BDUT1 can absorb the light. The spectrum detection circuit RL1 then detects optical response information of the blood under test BDUT1. In this embodiment, the wavelength range or the three wavelengths (including a first wavelength, a second wavelength, and a third wavelength) of the light may be, for example, between 350 nm and 430 nm (i.e., between wavelength ranges of ultraviolet light and visible light), or multiple wavelengths of light can be selected for illumination, such as: light having a wavelength of 382 nm, 406 nm, or 410 nm. In addition, the first wavelength, the second wavelength, and the third wavelength of light in this embodiment are mainly configured in a wavelength range between ultraviolet light (from 10 nm to 400 nm) or visible light (from 400 nm to 700 nm). Therefore, the light source providing circuit LP1 in the present disclosure can be implemented using existing commercially available electronic products. That is, the optical response information can be obtained by using one light source providing circuit LP1 to provide light in a range of wavelengths or by using three light source providing circuits LP1 to provide lights of three wavelengths to the blood under test, such that the optical response information is obtained from a corresponding optical response information receiver or a corresponding spectrum detection circuit RL1.

Referring to FIG. 4, in step S130, the first wavelength is an isosbestic point (a wavelength range C1 or a wavelength range C2). That is, in the first wavelength, after spectral detection is performed on the optical response information corresponding to different percentages of glycated hemoglobin, the optical response information corresponding to different glycated hemoglobin percentages are the closest to each other. In detail, the calculation for the first wavelength is to calculate a standard deviation of each of wavelength points and select a wavelength point having the smallest standard deviation, and the smaller the standard deviation is, the smaller a gap between data is. Afterwards, an average value of each of the wavelength points is calculated, the standard deviation is then calculated for all the average values of each of the wavelength points, and a wavelength point having the smallest standard deviation is finally selected. Next, a normalization and correction process is performed on the optical response information of the blood under test according to the optical response information of the first wavelength. In this embodiment, the first wavelength is within a wavelength range of from 584 nm to 588 nm (the wavelength range C1) or within a wavelength range of from 402 nm to 407 nm (the wavelength range C2), such as, but not limited to wavelengths of 586.5 nm and 405.8 nm, and the present disclosure is not limited thereto. The optical response information includes absorption spectrum intensity, diffusion spectrum intensity, absorbance, or various types of normalized optical response information, and is not limited in the present disclosure.

Referring to FIG. 5, in step S140 of this embodiment, the second wavelength can be at least within a wavelength range of from 565 nm to 584 nm (a wavelength range A1), a wavelength range of from 525 nm to 555 nm (a wavelength range A2), or a wavelength range of from 407 nm to 425 nm (a wavelength range A3). In addition, the third wavelength is at least within a wavelength range of from 590 nm to 610 nm (a wavelength range B1), or a wavelength range of from 355 nm to 383 nm (a wavelength range B2).

Referring to FIG. 6A and FIG. 6B, in this embodiment, in the second wavelength (within the wavelength range A1, the wavelength range A2, or the wavelength range A3), the optical response information is negatively correlated with the glycated hemoglobin percentage, that is, the stronger the corresponding optical response information is, the lower the glycated hemoglobin percentage becomes. However, in the third wavelength (within the wavelength range B1 or the wavelength range B2), the optical response information is positively correlated with the glycated hemoglobin percentage, that is, the stronger the optical response information is, the higher the glycated hemoglobin percentage becomes. That is, in this embodiment, the glycated hemoglobin percentage detection method uses the first wavelength for normalization and correction, and then uses the correlation between the respective intensity and percentage of the second wavelength (in which the intensity and percentage are negatively correlated) and the third wavelength (in which the intensity and percentage are positively correlated) to effectively detect the glycated hemoglobin percentage accurately.

Referring to FIG. 5, the optical response information corresponding to each of the glycated hemoglobin percentages can be distinctively and clearly distinguished. Therefore, the glycated hemoglobin percentage in the blood under test BDUT1 can be calculated based on the normalized optical response information. The normalized optical response information of different glycated hemoglobin percentages can be configured as a normalized optical response information and glycated hemoglobin content percentage comparison table for subsequent comparisons.

In this embodiment, the first wavelength is within a wavelength range of from 584 nm to 588 nm (the wavelength range C1), and a selected peak frequency is the wavelength of 586.5 nm. The second wavelength is within a wavelength range of from 565 nm to 584 nm (the wavelength range A1), and a selected peak frequency is the wavelength of 576.5 nm. The third wavelength is within a wavelength range of from 590 nm to 610 nm (the wavelength range B1), and a selected peak frequency is the wavelength of 601.2 nm. Table 1 below shows the performance of the first wavelength of 586.5 nm, the second wavelength of 576.5 nm, and the third wavelength of 601.2 nm on various model indicators.

TABLE 1

| Wavelength | | Root | | Coeffi- | | |
|---|---|---|---|---|---|---|
| combination/ | Mean | mean | Mean | cient of | Intraclass | |
| model index | square | square | absolute | determi- | correlation | |
| 576.5 nm + | error | error | error | nation | coefficient | |
| 601.2 nm | (MSE) | (RMSE) | (MAE) | (R2) | (ICC) | CI95% |
| blood sample | 0.176 | 0.419 | 0.359 | 0.872 | 0.964 | [0.92, |
| | | | | | | 0.98] |

In Table 1 above, an intraclass correlation coefficient ICC is used to evaluate the consistency and reliability between the results of this method and the existing standard results of high-performance liquid chromatography (HPLC).

In Table 1 above, the glycated hemoglobin percentage can be calculated by using the following formulas.

A formula for HbA1c includes:

$$HbA 1 c ( \% ) = \frac{{C}_{HbA 1 c}}{{C}_{HbA 1 c} + {C}_{HbA 0}} \times 100 \%$$

(in the definition of glycated hemoglobin percentage, HbA0 is non-glycated hemoglobin).

Formula 1 includes: HbA1c (%)=a*R²+b*R+c.

Here, the symbol R is a proportional component; the coefficient a, the coefficient b, and the coefficient c can be adjusted according to a user experience and are not limited in the present disclosure. The value A is the absorbance, and the value A (absorbance) can be calculated according to the Beer-Lambert law. That is, the symbol R in the above-mentioned Formula 1 can be calculated using an alternating current component A_{AC} and a direct current component A_{DC} of the absorbance A. Furthermore, when light of at least one illumination wavelength or light of a predetermined wavelength in this embodiment illuminates the human body part, an initial light intensity of light of the at least one illumination wavelength is a light intensity I₀. When the light having the light intensity I₀ illuminates tissue, skin, and blood in the blood vessel, the light will be absorbed to retain a light intensity I₁. When the light having the light intensity I₀ illuminates tissue, skin, blood in the blood vessel, cardiac contraction, or changes in arterial blood, the light having the light intensity I₀ will be absorbed to retain a light intensity I₂.

R can be

$$\frac{{AC}_{λ1} / {DC}_{λ1}}{A {C}_{λ2} / {DC}_{λ2}} ,$$

in which AC_{λ} is the alternating current component at a wavelength λ, that is, the change in the light intensity caused by changes in the arterial blood due to heart contraction; and DC_{λ} is the direct current component at the wavelength λ, that is, a baseline part of the light intensity including the absorption and diffuse reflection of light by parts such as tissue, skin, and blood in the blood vessel.

When converted into absorbance A, the alternating current component (A_{AC}) of absorbance A is the change in light intensity caused by changes in the arterial blood due to cardiac contraction; and the direct current component (A_{DC}) of absorbance A represents the baseline part of light intensity, including the absorption and diffuse reflection of light by parts such as tissue, skin, and blood in the blood vessel. That is, the symbol R may be equal to (A_{ACλ1}/A_{DCλ1})/(A_{ACλ2}/A_{DCλ2}).

R can be

$$\frac{1 - {10}^{- k 1 \cdot {A}_{λ1}^{corr}}}{1 - {10}^{- k 2 \cdot {A}_{λ2}^{corr}}} ,$$

in which the proportional coefficient k1 and the proportional coefficient k2 are adjusted according to the wavelength selected by the user and user experience and are not limited in the present disclosure, and

$${A}_{λ}^{corr}$$

is a corrected absorbance at the wavelength λ.

In addition, the symbol R can also be expressed by a light intensity I, that is, the symbol R can also be equal to (I_{ACλ1}/I_{DCλ1})/(I_{ACλ2}/I_{DCλ2}). Furthermore, the absorbance A can be calculated using the Beer-Lambert formula of:

$$A = - {log}_{10} \frac{{I}_{t}}{{I}_{0}} = ε * 1 * c ;$$

in which, A indicates: an absorbance; I₀ indicates: an incident light intensity; Iₜ indicates: an emitted light intensity; symbol ε indicates: an absorption coefficient (L×g⁻¹×cm⁻¹); symbol l indicates: an optical path length, which is a thickness (cm) of an absorbing medium; and symbol c indicates a percentage of light-absorbing material, denominated in g/L.

When the above-mentioned formulas are incorporated for calculation, the symbol R of Formula 1 can be obtained. Then the symbol R and Formula 1 are used to obtain the percentage value of glycated hemoglobin (HbA1c) in Formula 1.

In this embodiment, the second wavelength includes three wavelength ranges, and the third wavelength includes two wavelength ranges. With the inclusion of the first wavelength, the spectrum detection circuit RL1 (i.e., a light sensor) that is sensitive to three wavelength ranges is used. Since only one combination of the first wavelength, the second wavelength, and the third wavelength is provided in this embodiment, other wavelength range combinations are further provided for reference in the following descriptions.

### Second Embodiment

In this embodiment, the first wavelength is within the wavelength range of from 584 nm to 588 nm (the wavelength range C1), and the selected peak frequency is the wavelength of 586.5 nm. The second wavelength is within the wavelength range of from 525 nm to 555 nm (the wavelength range A2), and the selected peak frequency is the wavelength of 541.5 nm. The third wavelength is within the wavelength range of from 590 nm to 610 nm (the wavelength range B1), and the selected peak frequency is the wavelength of 601.2 nm. Table 2 below shows the performance of the second wavelength at 541.5 nm and the third wavelength at 601.2 nm on various model indicators.

TABLE 2

| Wavelength | | Root | | Coeffi- | | |
|---|---|---|---|---|---|---|
| combination/ | Mean | mean | Mean | cient of | Intraclass | |
| model index | square | square | absolute | determi- | correlation | |
| 541.5 nm + | error | error | error | nation | coefficient | |
| 601.2 nm | (MSE) | (RMSE) | (MAE) | (R2) | (ICC) | CI95% |
| blood sample | 0.165 | 0.406 | 0.348 | 0.881 | 0.967 | [0.92, |
| | | | | | | 0.99] |

### Third Embodiment

In this embodiment, the first wavelength is within the wavelength range of from 584 nm to 588 nm (the wavelength range C1), and the selected peak frequency is the wavelength of 586.5 nm. The second wavelength is within the wavelength range of from 407 nm to 425 nm (the wavelength range A3), and the selected peak frequency is the wavelength of 413.3 nm. The third wavelength is within the wavelength range of from 590 nm to 610 nm (the wavelength range B1), and the selected peak frequency is the wavelength of 601.2 nm. Table 3 below shows the performance of the second wavelength at 413.3 nm and the third wavelength at 601.2 nm on various model indicators.

TABLE 3

| Wavelength | | Root | | Coeffi- | | |
|---|---|---|---|---|---|---|
| combination/ | Mean | mean | Mean | cient of | Intraclass | |
| model index | square | square | absolute | determi- | correlation | |
| 541.5 nm + | error | error | error | nation | coefficient | |
| 601.2 nm | (MSE) | (RMSE) | (MAE) | (R2) | (ICC) | CI95% |
| blood sample | 0.143 | 0.379 | 0.314 | 0.896 | 0.973 | [0.93, |
| | | | | | | 0.99] |

### Fourth Embodiment

In this embodiment, the first wavelength is within the wavelength range of from 402 nm to 407 nm (the wavelength range C2), and the selected peak frequency is the wavelength of 405.8 nm. The second wavelength is within the wavelength range of from 407 nm to 425 nm (the wavelength range A3), and the selected peak frequency is the wavelength of 409.7 nm. The third wavelength is the wavelength range of from 355 nm to 383 nm (the wavelength range B2), and the selected peak frequency is the wavelength of 381.6 nm. In this embodiment, the second wavelength and the third wavelength are relatively close to each other.

TABLE 4

| Wavelength | | Root | | Coeffi- | | |
|---|---|---|---|---|---|---|
| combination/ | Mean | mean | Mean | cient of | Intraclass | |
| model index | square | square | absolute | determi- | correlation | |
| 381.6 nm + | error | error | error | nation | coefficient | |
| 409.7 nm | (MSE) | (RMSE) | (MAE) | (R2) | (ICC) | CI95% |
| blood sample | 0.176 | 0.419 | 0.333 | 0.873 | 0.965 | [0.92, |
| | | | | | | 0.98] |

### Fifth Embodiment

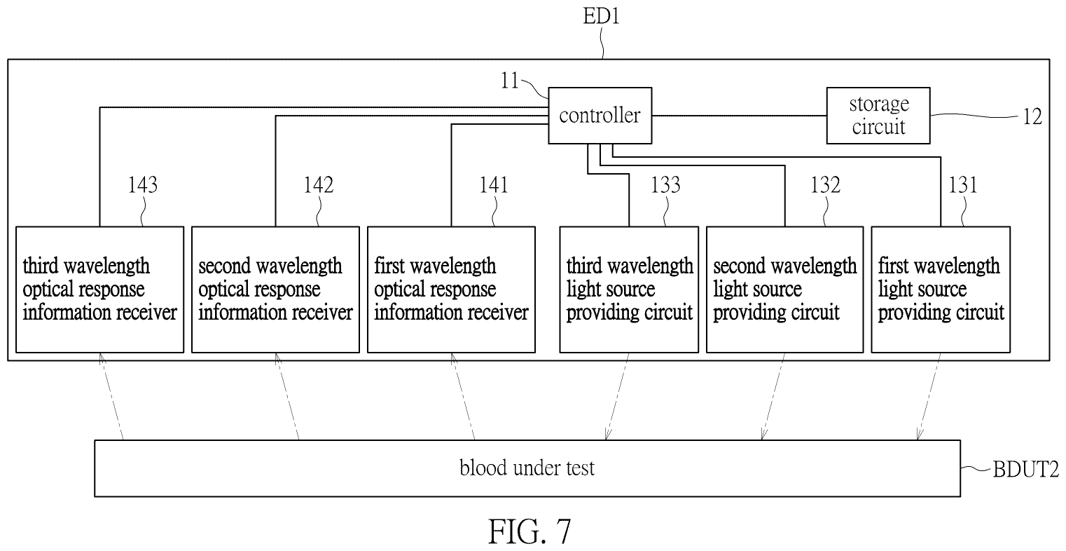

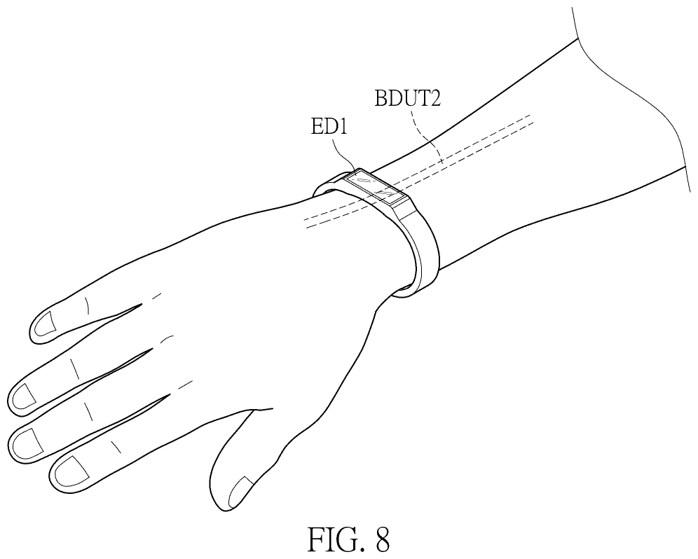

Referring to FIG. 7 and FIG. 8, FIG. 7 is a schematic diagram of a user wearing an electronic device according to a fifth embodiment of the present disclosure, and FIG. 8 is a functional block diagram of the electronic device according to the fifth embodiment of the present disclosure.

In this embodiment, an electronic device ED1 is provided for detecting the percentage of a blood component (e.g., glycated hemoglobin or blood sugar) of a blood under test BDUT2. The electronic device ED1 includes a smartphone, a tablet computer, a wearable electronic device, a medical device, or a non-invasive medical instrument. In this embodiment, the electronic device ED1 is a smart-watch or a smart-bracelet that can be worn on the wrist of a user. In addition, the electronic device ED1 can be a spectrometer, and the blood under test BDUT2 can be a blood sample. The blood under test BDUT2 is the blood content extracted from a user.

The electronic device ED1 includes a controller 11, a storage circuit 12, a first wavelength light source providing circuit 131, a second wavelength light source providing circuit 132, a third wavelength light source providing circuit 133, a first wavelength optical response information receiver 141, a second wavelength optical response information receiver 142, and a third wavelength optical response information receiver 143.

The storage circuit 12, the first wavelength light source providing circuit 131, the second wavelength light source providing circuit 132, the third wavelength light source providing circuit 133, the first wavelength optical response information receiver 141, the second wavelength optical response information receiver 142, and the third wavelength optical response information receiver 143 are connected to the controller 11. The controller 11 can also be connected to a server (not shown) or other electronic devices (not shown) through a communication circuit (not shown).

When the blood under test BDUT2 is disposed on one side of the light source providing circuit 13, the first wavelength light source providing circuit 131, the second wavelength light source providing circuit 132, and the third wavelength light source providing circuit 133 respectively provide lights of three wavelengths to the blood under test BDUT2, and the first wavelength optical response information receiver 141, the second wavelength optical response information receiver 142, and the third wavelength optical response information receiver 143 respectively receive optical response information of the blood under test BDUT2 corresponding to the three wavelengths. In this embodiment, the first wavelength light source providing circuit 131, the second wavelength light source providing circuit 132, and the third wavelength light source providing circuit 133 simultaneously provide light of the first wavelength, light of the second wavelength, and light of the third wavelength to the blood under test BDUT2. In other embodiments, the first wavelength light source providing circuit 131, the second wavelength light source providing circuit 132, and the third wavelength light source providing circuit 133 sequentially provide light of the first wavelength, light of the second wavelength, and light of the third wavelength to the blood under test BDUT2 according to an emission sequence. Similarly, the first wavelength optical response information receiver 141, the second wavelength optical response information receiver 142, and the third wavelength optical response information receiver 143 can simultaneously receive optical response information of all the wavelengths, and then the optical response information pass through a filter for signal analysis. The first wavelength optical response information receiver 141, the second wavelength optical response information receiver 142, and the third wavelength optical response information receiver 143 can also be sequentially activated according to the emission sequence of the light source providing circuits and respectively receive the optical response information corresponding to the first wavelength, the optical response information corresponding to the second wavelength, and the optical response information corresponding to the third wavelength. In addition, the first wavelength optical response information receiver 141, the second wavelength optical response information receiver 142, and the third wavelength optical response information receiver 143 only need to be positioned to receive scattered light after the light of the first wavelength, the light of the second wavelength, and the light of the third wavelength are emitted to the blood under test BDUT2, and positions of the receivers are not limited in the present disclosure.

The optical response information of three wavelengths of the blood under test BDUT2 detected by the first wavelength optical response information receiver 141, the second wavelength optical response information receiver 142, and the third wavelength optical response information receiver 143 is transmitted to the controller 11. The controller 11 selects an optical response information corresponding to the first wavelength (for example, the wavelength range C1 or the wavelength range C2) of the blood under test BDUT2 to perform a normalization and correction process, so as to obtain the normalized optical response information of the blood under test BDUT2.

In this embodiment, the lights of the first wavelength, the second wavelength, and the third wavelength or the light of a predetermined wavelength can be, for example, light having a wavelength of between 350 nm and 430 nm (the wavelength range between ultraviolet light and visible light). The lights of the first wavelength, the second wavelength, and the third wavelength can also be selected from light of multiple wavelengths, for example, light of wavelengths of 382 nm, 406 nm, or 410 nm for illumination. In addition, the lights of the first wavelength, the second wavelength, and the third wavelength in this embodiment are mainly configured to be within the wavelength range between ultraviolet light (from 10 nm to 400 nm) or visible light (from 400 nm to 700 nm).

The first wavelength is the isosbestic point (the wavelength range C1). In this embodiment, the first wavelength is within the wavelength range of from 584 nm to 588 nm (the wavelength range C1), such as but not limited to the wavelength of 586.5 nm, and the present disclosure is not limited thereto. The controller 11 performs normalization for the optical response information of the blood under test BDUT2 of the user according to the optical response information of the first wavelength. Then, the controller 11 selects a group of the second wavelength (the wavelength range of from 565 nm to 584 nm, i.e., the wavelength range A1; the wavelength range of from 525 nm to 555 nm, i.e., the wavelength range A2; or the wavelength range of from 407 nm to 425 nm, i.e., the wavelength range A3) and the third wavelength (the wavelength range of from 590 nm to 610 nm, i.e., the wavelength range B1; or the wavelength range of from 355 nm to 383 nm, i.e., the wavelength range B2), and performs a percentage test on the glycated hemoglobin of the blood under test BDUT2 of the user.

In this embodiment, in the second wavelength (within the wavelength range A1, the wavelength range A2, and the wavelength range A3), the optical response information value is negatively correlated with the percentage of blood components. That is, the stronger the optical response information value is, the lower the blood component percentage becomes. However, in the third wavelength (within the wavelength range B1 and the wavelength range B2), the optical response information is positively correlated with the glycated hemoglobin percentage. That is, the stronger the optical response information value is, the higher the glycated hemoglobin percentage becomes. In this embodiment, the controller 11 of the electronic device ED1 can use the first wavelength to perform normalization and correction, and reuse the correlation between the respective intensity and percentage of the second wavelength (in which the intensity and percentage are negatively correlated) and the third wavelength (in which the intensity and percentage are positively correlated) to effectively detect the glycated hemoglobin percentage in the blood under test of the body of the user accurately.

Furthermore, the storage circuit 12 stores the normalized optical response information and glycated hemoglobin content percentage comparison table. The normalized optical response information and glycated hemoglobin content percentage comparison table is configured by using the normalized optical response information of different percentages of glycated hemoglobin.

In addition, the controller 11 can store the glycated hemoglobin percentage information in the storage circuit 12 or transmit the glycated hemoglobin percentage information to a server (not shown) or an electronic device (not shown) through a communication circuit (not shown).

The controller 11 is a central processing unit (CPU), an application-specific integrated circuit (ASIC), a graphics processing unit (GPU), or a microprocessor (MCU).

The storage circuit 12 is a flash memory, a read-only memory, a programmable read-only memory, an electrically rewritable read-only memory, an erasable programmable read-only memory, or an electrically erasable programmable read-only memory.

The communication circuit (not shown) may include a wired communication member (not shown) and a wireless communication member (not shown). The wired communication member (not shown) can also be disposed independently to communicate with the server (not shown) and receive control signals from the server (not shown) or data in the database of the server (not shown). When the communication circuit (not shown) is a wireless communication member, the communication circuit (not shown) can be an Wi-Fi® communication member, a Bluetooth® communication member, a Zigbee® communication member, a LoRa® communication member, a Sigfox™ communication member, or an NB-IoT communication member.

### Beneficial Effects of the Embodiments

One of the beneficial effects of the present disclosure is that, in the glycated hemoglobin percentage detection method and an electronic device using the method provided by the present disclosure, common ultraviolet and visible light wavelength ranges can be used for spectral detection, and a comparison process of glycated hemoglobin percentage can be simplified; therefore costs are effectively lowered, and the amount of calculations required by the electronic device to determine the glycated hemoglobin percentage can be significantly reduced.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A glycated hemoglobin percentage detection method, comprising:
obtaining optical response information of a blood under test;
performing a normalization and correction process based on the optical response information of the blood under test of a first wavelength to obtain normalized optical response information of the blood under test; and
selecting normalized optical response information of a second wavelength and normalized optical response information of a third wavelength from the normalized optical response information of the blood under test for analysis to determine a glycated hemoglobin percentage in the blood under test.

2. The glycated hemoglobin percentage detection method according to claim 1, wherein the optical response information of the blood under test is obtained by using a light source providing circuit to provide light in a wavelength range or using three light source providing circuits to provide light in three wavelengths to the blood under test and obtaining through a corresponding optical response information receiver.

3. The glycated hemoglobin percentage detection method according to claim 2, wherein the blood under test is a blood sample or a blood content present in a human body part, and the human body part at least includes a finger and a wrist.

4. The glycated hemoglobin percentage detection method according to claim 3, wherein the normalized optical response information of the second wavelength and the normalized optical response information of the third wavelength are compared with a normalized optical response information and glycated hemoglobin content percentage comparison table to determine the glycated hemoglobin percentage in the blood under test.

5. The glycated hemoglobin percentage detection method according to claim 4, wherein the wavelength of the light provided by the light source providing circuit is between an ultraviolet wavelength range and a visible light wavelength range.

6. The glycated hemoglobin percentage detection method according to claim 5, wherein the first wavelength is within a wavelength range of from 580 nm to 590 nm and a wavelength of from 395 nm to 410 nm, the second wavelength is at least within a wavelength range of from 405 nm to 425 nm, a wavelength range of from 525 nm to 555 nm, or a wavelength range of from 565 nm to 585 nm, and the third wavelength is at least within a wavelength range of from 590 nm to 610 nm, or a wavelength range of from 355 nm to 383 nm.

7. The glycated hemoglobin percentage detection method according to claim 6, wherein the normalization and correction process includes dividing the optical response information of all wavelengths among the optical response information by the optical response information of the first wavelength.

8. An electronic device using a glycated hemoglobin percentage detection method for detecting a glycated hemoglobin percentage of a blood under test, the electronic device comprising:
a controller;
a storage circuit connected to the controller;
a first wavelength light source providing circuit connected to the controller;
a second wavelength light source providing circuit connected to the controller;
a third wavelength light source providing circuit connected to the controller;
a first wavelength optical response information receiver connected to the controller;
a second wavelength optical response information receiver connected to the controller; and
a third wavelength optical response information receiver connected to the controller;
wherein, when the first wavelength light source providing circuit emits light of a first wavelength to a blood under test, the second wavelength light source providing circuit emits light of a second wavelength to the blood under test, and the third wavelength light source providing circuit emits light of a third wavelength to the blood under test, the first wavelength optical response information receiver receives optical response information of the blood under test corresponding to the first wavelength, the second wavelength optical response information receiver receives optical response information of the blood under test corresponding to the second wavelength, and the third wavelength optical response information receiver receives optical response information of the blood under test corresponding to the third wavelength;
wherein the controller performs a normalization and correction process according to the optical response information corresponding to the first wavelength, and the controller performs the normalization and correction process for the optical response information corresponding to the second wavelength and the optical response information corresponding to the third wavelength according to the optical response information corresponding to the first wavelength, to obtain a normalized optical response information corresponding to the second wavelength and a normalized optical response information corresponding to the third wavelength for the blood under test;
wherein the controller analyzes the normalized optical response information corresponding to the second wavelength and the normalized optical response information corresponding to the third wavelength to determine a glycated hemoglobin percentage of the blood under test.

9. The electronic device according to claim 8, wherein the electronic device is a smartphone, a tablet computer, a wearable electronic device, a medical device, or a dedicated glycated hemoglobin detection device, and the blood under test is a blood content present in a human body part; wherein the electronic device provides light of at least one wavelength to illuminate the human body part, and receives the optical response information of the blood under test through the process of light penetration and light reflection.

10. The electronic device according to claim 9, wherein the controller compares the normalized optical response information of the second wavelength and the normalized optical response information of the third wavelength with a normalized optical response information and glycated hemoglobin content percentage comparison table stored in the storage circuit to determine the glycated hemoglobin percentage.
